(19) United States
(12) Patent Application Publication
(10) Pub. No.: US 2012/0174914 A1
(43) Pub. Date: Jul. 12, 2012

(54) Electronic vapor inhaling device

(76) Inventors: Nasser Pirshafiey, Thousand Oaks, CA (US); Edward Montezinos, Utrecht (NL); Eric Swerdloff, Beverly Hills, CA (US)

(21) Appl. No.: 13/374,666

(22) Filed: Jan. 5, 2012

Related U.S. Application Data
(60) Provisional application No. 61/460,782, filed on Jan. 8, 2011.

(51) Int. Cl. A61M 11/00

(52) U.S. Cl. 128/200.14

(57) ABSTRACT

An electronic vapor inhaling device has an outer housing that defines an internal cavity having a proximal end and a distal end. A mouthpiece is located at the proximal end. A liquid storage chamber is defined within the outer housing near its proximal end and a battery compartment containing a battery is located near the distal end. An atomizer is positioned between the liquid storage chamber and battery chamber and is in communication with an electronic circuit board. The electronic circuit board is configured to receive input from a unique identifier associated with the electrical resistance of the liquid in the liquid storage chamber and automatically adjust the power supplied to the atomizer. The atomizer rapidly heats the liquid that is injected into an air passageway when the user inhales through the mouthpiece, causing the liquid to vaporize and allow inhalation by the user.

### CROSS-REFERENCE TO RELATED APPLICATION

This application claims priority from provisional patent application Ser. No. 61/460,782, filed Jan. 8, 2011.

### STATEMENT REGARDING FEDERALLY SPONSORED RESEARCH OR DEVELOPMENT

Not Applicable.

### REFERENCE TO A “MICROFICHE APPENDIX”

Not Applicable.

### BACKGROUND

1. Field of the Inventions

The field of the invention relates to an electronic vapor inhaling device.

2. Background Information

Conventional smoking devices, including cigarettes, cigars and pipes, etc., are now known to be associated with a variety of problems. Most notably, such devices typically burn tobacco that includes a variety of substances including tar, for example, that are believed to be associated with an increased risk of health problems, including life-threatening diseases such as cancer. In addition, the burning of the tobacco naturally requires the use of a flame which consequently presents a significant fire risk in many environments. Also, the experience of others' smoke can also be unpleasant for third-parties, especially in public venues.

To address these and other problems associated with conventional smoking devices, smoking-substitute devices have been developed with the goal of eliminating the use of fire to burn tobacco, thereby eliminating the odorous and potentially unhealthy smoke that typically occurs, and also reducing the risk of accidental fires. Such devices typically make use of an electrically operated device that is configured to convert a liquid that contains nicotine or other substance that the user desires to inhale into a breathable vapor. However, current devices typically have numerous shortcomings including diminished battery life and power management issues, reliability issues, and the inability to efficiently adjust the voltage supplied to the atomizer to properly vaporize the liquid substrate. Accordingly, there is a need for an improved electronic smoking device to overcome the numerous shortcomings of current devices.

### SUMMARY OF THE INVENTION

Systems and methods for the formation and use of an electronic vapor inhaling device are disclosed. In one aspect, the electronic vapor inhaling device has an outer housing that defines an internal cavity having a proximal end and a distal end. A mouthpiece is located at the proximal end. A liquid storage chamber is defined within the outer housing near its proximal end and a battery compartment containing a battery is located near the distal end.

In another aspect, the electronic vapor inhaling device includes an atomizer positioned between the liquid storage chamber and battery chamber and is in communication with an electronic circuit board.

In another aspect, an air passageway is defined through the mouth piece, the liquid storage chamber, the atomizer, and the outer housing, allowing air to travel through the air passageway when the user inhales. The air passageway can traverse through the wall structure of the outer housing between the liquid storage chamber and the battery chamber. A portion of the air passageway between the atomizer and the outer housing is in communication with a low pressure chamber that is configured to have a lower air pressure than the inside of the liquid storage chamber when air travels through the air passageway, causing liquid to be injected from the liquid storage chamber into the air passageway.

In another aspect, the electronic vapor inhaling device can include a cartridge that contains liquid. The cartridge is inserted into the liquid storage chamber and can be disposable. The cartridge can include a unique identifier, such as a bar code or RFID tag, that is associated with the electrical resistance of the liquid stored within the cartridge.

In another aspect, the electronic circuit board is configured to receive input from a unique identifier associated with the electrical resistance of the liquid in the liquid storage chamber and automatically adjust the power supplied to the atomizer. The atomizer rapidly heats the liquid that is injected into an air passageway when the user inhales through the mouthpiece, causing the liquid to vaporize and allow inhalation by the user.

In another aspect, the electronic circuit board includes an air flow sensor that is configured to activate the atomizer when the air flow sensor detects air flow through the air passageway.

These and other features, aspects, and embodiments of the inventions are described below in the section entitled “Detailed Description of the Preferred Embodiments.”

### BRIEF DESCRIPTION OF THE DRAWINGS

Features, aspects, and embodiments of the inventions are described in conjunction with the attached drawings, in which:

FIG. 1 illustrates a perspective view of a vapor inhaling device in one example embodiment of the invention.

FIG. 2 illustrates a perspective view of a vapor inhaling device in another example embodiment of the invention.

FIG. 3 illustrates a perspective view of a vapor inhaling device in another example embodiment of the invention.

FIG. 4 illustrates a cross sectional view of the mouth piece of a vapor inhaling device in another example embodiment of the invention.

FIG. 5 illustrates a cross sectional view of the outer housing of a vapor inhaling device in another example embodiment of the invention.

FIG. 6 is an exploded view of a portion of the junction between the liquid storage chamber and battery compartment of a vapor inhaling device in another example embodiment of the invention.

FIG. 7 is a block diagram of the electrically powered control components of a vapor inhaling device in another example embodiment of the invention.

### DETAILED DESCRIPTION OF THE PREFERRED EMBODIMENTS

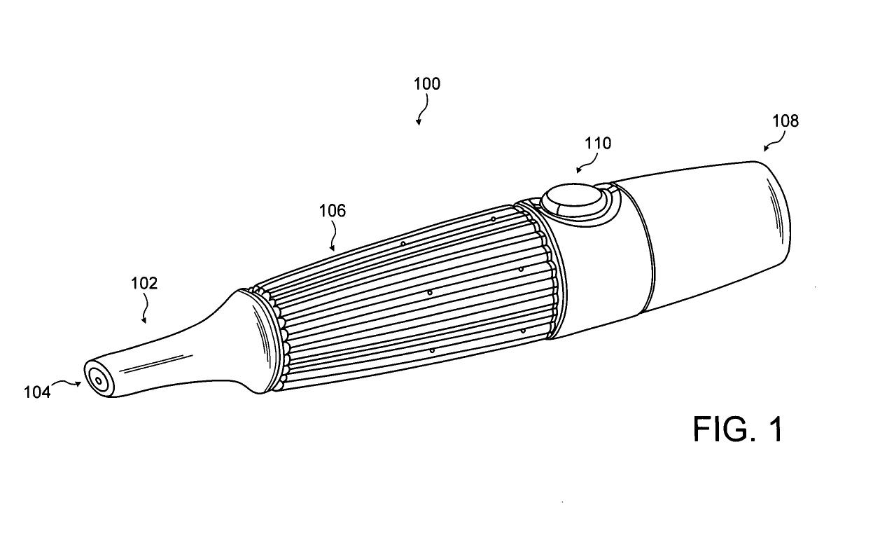

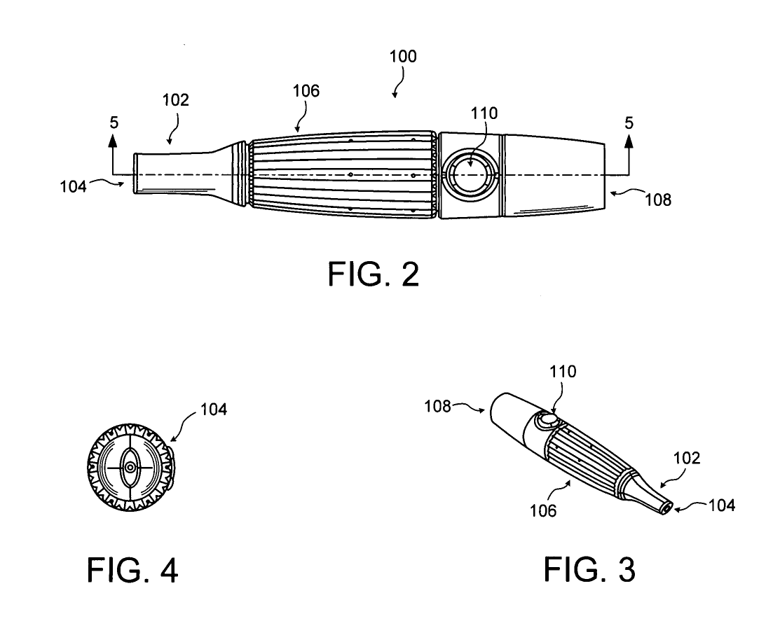

Referring to FIGS. 1-3, exemplary embodiments of vapor inhaling device 100 are illustrated. In one exemplary embodiment, vapor inhaling device 100 has an overall shape that is generally rod-like or tubular in nature. Vapor inhaling device 100 possesses a mouth piece 102 with mouth-end 104 that is coupled to outer housing 106 having distal end 108. Although the dimensions and shape of the outer housing 106 can vary, a representative outer housing is generally tubular in shape as illustrated.

Mouth piece 102 can be provided using a variety of materials. For example, mouth piece 102 can be provided from metal, wood or plastic. As illustrated in FIGS. 1-4, mouth end 104 of mouth piece 102 comprises an opening adapted for egress of an aerosol generated within the vapor inhaling device 100. The distal end of mouth piece 102 comprises a connection for removably connecting mouth piece 102 to outer housing 106 through a threaded connection or a snap-fit, for example. In use, this allows mouth piece 102 to be easily replaced as needed by one or more users and also provides a point of access to the inner chamber of outer housing 106.

Similarly, outer housing 106 can also be provided using a variety of materials, such as metal, wood or plastic, for example. In one exemplary embodiment, outer housing 106 possess an aesthetically pleasing design including a rigid portion formed by a plurality of raised ridges as illustrated that provides an improved gripping surface for the user. In another exemplary embodiment, switch 110 is disposed near the surface of outer housing 106, and as described in more detail below, switch 110 can be used to manually activate vapor inhaling device 100 in one embodiment. In yet another embodiment, switch 110 can be omitted and replaced instead with an ornamental element such as a cosmetic jewel, for example.

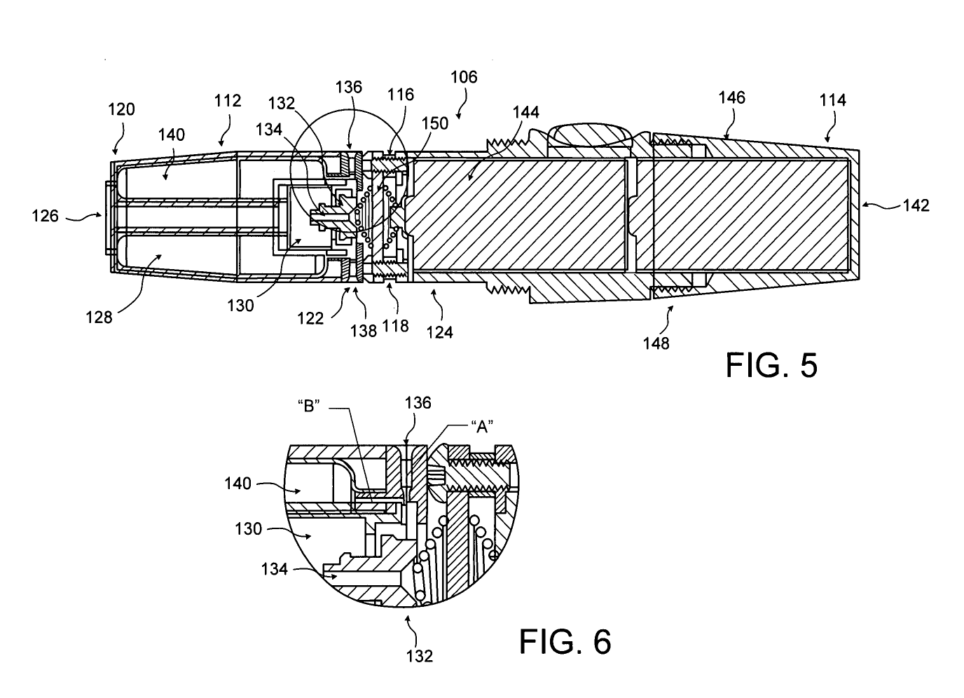

Turning now to FIG. 5, outer housing 106 is illustrated in a cross sectional view. As shown, it can be appreciated that in the exemplary embodiment illustrated in FIG. 5, outer housing 106 comprises wall structure that defines liquid storage chamber 112 and battery compartment 114. In the exemplary embodiment shown in FIG. 5, liquid storage chamber 112 and battery compartment 114 are formed as two separate pieces and joined together with one or more fasteners such as screws 116 and 118. Alternatively, liquid storage chamber 112 and battery compartment 114 can be coupled through a friction fit, press-fit, bayonet fit, snap fit, or a threaded connection, for example.

As discussed in detail below and illustrated in FIGS. 5 and 6, the components and internal structure of mouth piece 102 and liquid storage chamber 112 create a continuous air passageway that begins at the proximal end of mouthpiece 102, traverses through liquid storage chamber 112, and is in communication with the external environment outside of liquid storage chamber 112 near the junction of liquid storage chamber 112 and battery compartment 114.

More specifically, liquid storage chamber 112 has proximal end 120 and distal end 122, with the proximal end configured to be coupled with distal end of mouth piece 102 and distal end 122 configured to be coupled with the proximal end 124 of battery compartment 114. The wall structure of the proximal end 120 of liquid storage chamber 112 defines an opening 126 that is configured to correspond to the opening in mouth piece 102 to allow the passage of air and aerosol from the liquid storage chamber 112 through mouth piece 102 to the user. In addition, opening 126 is in communication with air passage 128 that is defined by wall structure within liquid storage chamber 112 and terminates at atomizer chamber 130 formed from wall structure near the distal end of liquid storage chamber 112.

As illustrated in FIG. 6 which is an exploded view of a portion of the junction between liquid storage chamber 112 and battery compartment 114, atomizer chamber 130 defines a hollow space that contains atomizer 132, the function and operation of which will be discussed in more detail below. Atomizer 132 defines internal atomizer air passage 134 that is in communication with atomizer chamber 130 at its proximal end and external air passages 136 and 138. External air passages 136 and 138 can be identical in structure, comprising wall structure that defines an air passageway that extends radially and longitudinally from internal atomizer air passage 134 to and through the external wall structure of liquid storage chamber 112, thereby completing the continuous air passageway from mouth piece 102 through the liquid storage chamber 112 to the external environment. In another preferred embodiment, a plurality of external air passages are provided, each extending from internal atomizer air passage 134 to and through the external wall structure of liquid storage chamber 112.

As illustrated in detail in FIG. 6, external air passage 136 defines a first area where the air passage has an area of increased volume identified as region “A” and a second region of increased volume identified as region “B”. When the user inhales through mouthpiece 102, airflow through the portion of air passage 136 identified as region “A” creates a temporary low pressure condition in the portion of air passage 136 identified as region “B”. This change in pressure causes liquid to be injected from cartridge 140 into the air stream passing through air passage 136 which in turn moves into atomizer 132. In another exemplary embodiment, air passage 138 is also provided and includes the same two types of regions of increased volume as just discussed with reference to air passage 136, thereby also causing liquid to be injected from cartridge 140 into the air stream passing through air passage 138 in response to the region of low pressure created when the user inhales through mouthpiece 102. While not illustrated, persons of ordinary skill in the art can appreciate that the same configuration described above can be repeated for each of the plurality of air passages that extend radially and longitudinally from atomizer 132 to and through the external wall structure of liquid storage chamber 112.

As illustrated in FIG. 5, liquid storage chamber 112 defines a hollow region between the external wall structure and the air passage 128. The hollow region is adapted to receive cartridge 140. Cartridge 140 defines an opening that is in communication with air passageway 136 in the area identified by reference “B.” As the user inhales, the area of low pressure created in area “B” causes liquid to be expelled from cartridge 140 into air passageway 136.

In another embodiment, cartridge 140 is a refillable tank that is configured to store the liquid, such as a vegetable glycerine or propylene glycol based liquid, that will ultimately be vaporized and inhaled by the user. In exemplary embodiments, cartridge 140 can be made from plastic, glass, or metal. Cartridge 140 can be configured to be refilled by the user by using a syringe, eye drop, or similar device.

In another embodiment, cartridge 140 is a disposable tank that can be made from plastic, glass or metal. In another embodiment, cartridge 140 can further comprise an internal bag that is configured to deflate as its liquid contents are expelled, thereby providing maximum ejection of the liquids within the tank and maximizing the amount of liquid that is used. After the contents of the disposable cartridge 140 have been used, the user can remove it and then insert a new cartridge. Cartridge 140 can be removed from liquid storage chamber by disconnecting liquid storage chamber 112 from battery storage compartment 114. In yet another embodiment, atomizer 132 is provided as a component of the cartridge 140, such that a new atomizer 132 is provided whenever the user replaces cartridge 140.

Turning now to battery compartment 114, wall structure defines a region within battery compartment 114 that contains an electric power source, such as at least one battery 142. The battery typically is maintained in place by the generally tubular geometry of the wall structure. In another exemplary embodiment, a second battery 144 is also provided within battery compartment 114, which can be modified to include different numbers and types of batteries to achieve a desired voltage source.

In another exemplary embodiment, the wall structure of battery compartment 114 includes end cap 146 that can be removably connected to the proximal portion of battery compartment 114 through a variety of connections such as the tongue and groove connection 148 illustrated in FIG. 5, or through a threaded connection, snap fit, bayonet fit, or the like. This removable connection allows for simple replacement of the batteries located in battery storage compartment 114.

The vapor inhaling device 100 incorporates various electrically powered control components 150 that manage the operation of the device. For an exemplary embodiment, the control components 150 are positioned near or at the junction of liquid storage chamber 112 and battery storage compartment 114 as shown. The electrically powered control components are configured to provide the proper amount of wattage to atomizer 132 to cause the liquid to be heated to the proper temperature to provide a usable, inhalable vapor to the user. Atomizer 132 comprises a body with a filament that heats up when a predetermined wattage is applied to the atomizer. The rapid heating of the liquid by the atomizer causes the liquid to vaporize, thereby allowing for inhalation by the user.

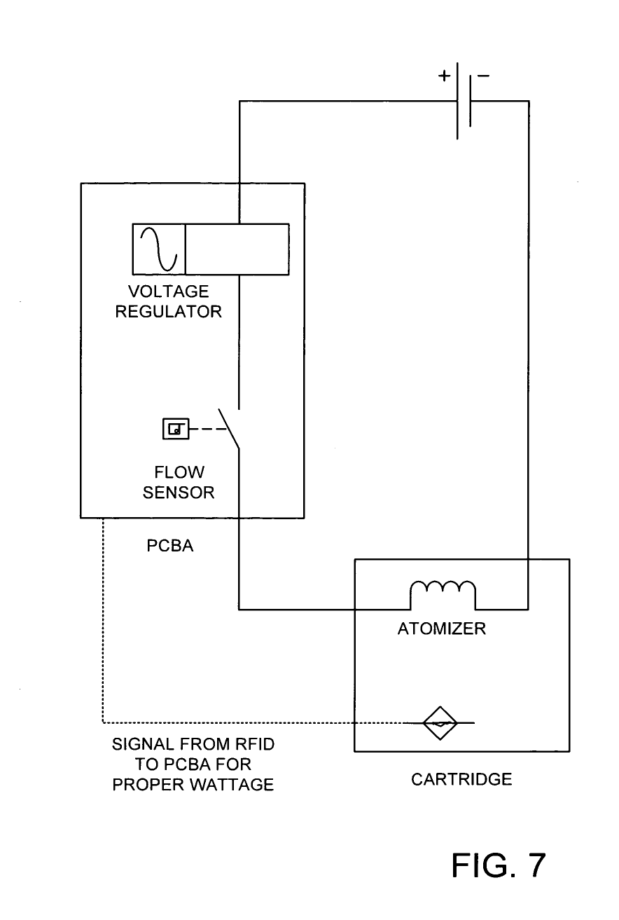

Typically, the electrically-powered control components 150 include either hardware implementations or preferably a printed circuit board assembly (PCBA) that is configured to control the operation of smoking device 100. Exemplary circuits that can be included in the controller 150 are set forth in FIG. 7, and the electrically-powered control components 150 are powered by the battery.

In one exemplary embodiment, the electrically-powered control components 150 are configured to provide a constant and optimum wattage based on the overall system resistance, thereby maximizing battery life. In another embodiment, the electrically-powered control components 150 can detect and identify different liquids in different cartridges 140 that have different concentrations of substances that users desire to inhale, for example. The electrically-powered control components 150 are configured to adjust the wattage supplied to the atomizer 132 by reading an RFID tag or barcode, for example, that is disposed on a cartridge 140 and uniquely associated with a certain liquid in a particular cartridge, or by recognizing the atomizer by measuring its resistance. The electrically-powered control components 150 are then configured to automatically adjust the wattage supplied to the atomizer 132 to provide the optimal wattage to atomizer 132 for that particular cartridge. More specifically, depending on a particular type and concentration of liquid, the proper wattage must be applied because too much wattage results in burning the liquid while too little wattage fails to vaporize the liquid and the liquid ends up reaching the users mouth. In an alternative embodiment, the user can manually adjust the wattage supplied to atomizer 132 to provide a desired vapor.

In yet another embodiment, the electrically-powered control components 150 include a flow sensor that is configured to detect air flow caused by the user inhaling. Upon detecting airflow, the flow sensor is configured to cause the electrically-powered control components to provide power to the atomizer 132. In an alternative embodiment, an additional main switch can be added to turn the circuit off during extended periods of planned non-use in order to further conserve battery power.

In yet another embodiment, the electrically-powered control components 150 can omit the flow sensor, and instead rely on a switch 110 that is manually activated by the user at the time the user desires inhale a vapor. When the manual switch is pressed, the atomizer is activated. In yet another embodiment, a combination of the flow sensor and manual switch can be implemented. That is, both the flow sensor and the manual switch must be activated in order for the atomizer to become energized. The former by the user inhaling through mouth piece 102 and the latter by manual operation by the user. If either the flow sensor or the switch are not activated, the atomizer is not energized.

While certain embodiments of the inventions have been described above, it will be understood that the embodiments described are by way of example only. Accordingly, the inventions should not be limited based on the described embodiments.

What is claimed:

1. An electronic vapor inhaling device comprising:
an outer housing having a proximal end and a distal end, the outer housing comprising wall structure defining an internal cavity;
a mouth piece coupled to the proximal end of the outer housing;
a liquid storage chamber formed by the wall structure of the outer housing near the proximal end of the outer housing;
a cartridge containing a liquid, the cartridge positioned in the liquid storage chamber;
a battery compartment formed by the wall structure of the outer housing near the distal end of the outer housing;
an atomizer chamber disposed between the liquid storage chamber and the battery compartment;
an atomizer positioned in the atomizer chamber;
an electronic circuit board coupled to the atomizer;
a battery positioned in the battery compartment, the battery in communication with the electronic circuit board; and
and an air passageway defined through the mouth piece, the liquid storage chamber, the atomizer, and the outer housing.

2. The electronic vapor inhaling device of claim one, wherein the air passageway traverses through the wall structure of the outer housing between the liquid storage chamber and the battery compartment.

3. The electronic vapor inhaling device of claim two, wherein the portion of the air passageway between the atomizer and the outer housing is in communication with a low pressure chamber.

4. The electronic vapor inhaling device of claim three, wherein the low pressure chamber is configured to have a lower air pressure than the inside of the cartridge when air travels through the air passageway, thereby causing liquid to be injected from the cartridge to the air passageway.

5. The electronic vapor inhaling device of claim four, wherein the cartridge further comprises an internal bag containing a liquid, wherein the internal bag is configured to deflate as the liquid is injected to the air passageway.

6. The electronic vapor inhaling device of claim four, wherein the cartridge further comprises a unique identifier disposed on the cartridge.

7. The electronic vapor inhaling device of claim six, wherein the unique identifier is associated with the electrical resistance of the liquid in the cartridge.

8. The electronic vapor inhaling device of claim seven, wherein the electronic circuit board further comprises a memory, wherein the memory stores the unique identifier and the electrical resistance of the liquid associated with the unique identifier.

9. The electronic vapor inhaling device of claim eight, wherein the electronic circuit board further comprises means for receiving input from the unique identifier.

10. The electronic vapor inhaling device of claim nine, wherein the electronic circuit board further comprises a processor.

11. The electronic vapor inhaling device of claim ten, wherein the processor is configured to:
receive the input from the unique identifier;
associate the unique identifier with the electrical resistance of the liquid in the cartridge from the memory; and
automatically adjust the power supplied from the battery to the atomizer based on the electrical resistance of the liquid in the cartridge.

12. The electronic vapor inhaling device of claim eleven, wherein the unique identifier comprises a bar code.

13. The electronic vapor inhaling device of claim eleven, wherein the unique identifier comprises an RFID tag.

14. The electronic vapor inhaling device of claim eleven, wherein the electronic circuit board further comprises an air flow sensor.

15. The electronic vapor inhaling device of claim fourteen, wherein the air flow sensor is configured to send a signal to activate the atomizer when the air flow sensor detects air flow through the air passageway.

16. The electronic vapor inhaling device of claim eleven, wherein the electronic circuit board further comprises a switch that is configured to activate the atomizer.

17. The electronic vapor inhaling device of claim eleven, wherein the cartridge is disposable and can be replaced by the user.

18. The electronic vapor inhaling device of claim seventeen, further comprising a plurality of cartridges configured to be placed in the liquid storage chamber, each cartridge containing a different liquid.

19. The electronic vapor inhaling device of claim eighteen, wherein each cartridge has a unique identifier associated with the electrical resistance of the liquid in the cartridge.

20. The electronic vapor inhaling device of claim nineteen, wherein the atomizer is formed as an integral part of the cartridge.
